(19) United States
(12) Patent Application Publication
(10) Pub. No.: US 2023/0310684 A1
(43) Pub. Date: Oct. 5, 2023

(54) DEVICE FOR DISINFECTING AN INTERIOR SPACE

(71) Applicants: Frank Rister, Hamburg (DE); Nils Bartling, Hamburg (DE); Wulfert Kirst, Hamburg (DE)

(72) Inventors: Frank Rister, Hamburg (DE); Nils Bartling, Hamburg (DE); Wulfert Kirst, Hamburg (DE)

(21) Appl. No.: 18/022,838

(22) PCT Filed: Aug. 23, 2021
(86) PCT No.: PCT/EP2021/073263

§ 371 Date: Feb. 23, 2023

(30) Foreign Application Priority Data
Aug. 24, 2020 (EP) 20192443.8

(51) Int. Cl. A61L 2/24; B64U 10/60; A61L 2/10

(52) U.S. Cl. CPC A61L 2/24; A61L 2/10; B64U 10/60; B64U 2101/70

(57) ABSTRACT

Device for disinfecting an interior space with a cart, which is configured to travel along a path through the interior space, and a germicidal light source, characterized by a drone, which has at least one electric drive for generating lift, wherein the germicidal light source is fastened to the drone and the drone is connected to the cart via an electrical line for supplying energy to the germicidal light source and the electric drive.

### BACKGROUND OF THE INVENTION

The invention relates to a device for disinfecting an interior space with a cart and a germicidal light source. The cart can travel, for example, through the aisle of an aircraft cabin and subject the surrounding surfaces, for example, the seats, to the germicidal light, thereby disinfecting them.

The document WO 2019/068189 A1 describes the disinfection of a vehicle cabin with stationary UV illumination.

The document US 2019/0030195 A1 shows stationary UV light sources for disinfecting an aircraft cabin. It is especially about a control of the individual light sources depending on the presence of persons in certain zones of the aircraft cabin.

The document US 2017/0290935 A1 describes disinfection of aisles and lavatories in aircraft cabins using UV light. The device used comprises a cart, on which a boom with a UV light source is arranged.

The document US 2016/0339133 A1 shows a mobile, battery-operated device that has a mercury or amalgam vapor lamp for generating UVC light. Use in an aircraft lavatory is proposed.

Devices for disinfecting an aircraft cabin are known from documents WO 2016/164362 A1 and WO 2016/164364 A1. They each comprise a cart, which is driven by hand or autonomously through an aisle of the aircraft and has extendable arms on both sides, on which UVC lights are arranged. A battery is located in the cart for supplying energy. Alternatively, an energy supply from a power grid via a cable is addressed.

The document WO 2018/164845 A1 shows a similarly built cart for disinfecting aircraft cabins with UVC light like the two previously discussed documents from the same applicant. The cart is intended to be pushed manually through an aisle of the aircraft cabin by an operating person and has a protective shield intended to shield the operating person from the UVC radiation.

A remote-controlled camera drone equipped with 36 UVC light-emitting diodes is known from the company brochure “Aertos 120-UVC” from the company Digital Aerolus. The maximum flight time is intended to be 10 minutes.

### BRIEF SUMMARY OF THE INVENTION

Starting from this, the object of the invention is to provide a device for disinfecting an interior space that can be used sufficiently robustly, effectively, and flexibly for commercial use.

The device serves to disinfect an interior space and has
a cart, which is configured to travel along a path through the interior space,
a germicidal light source, and
a drone, which has at least one electric drive for generating lift, wherein
the germicidal light source is fastened to the drone and
the drone is connected to the cart via an electrical line for supplying energy to the germicidal light source and the electric drive.

The interior space can be, for example, a hotel room, a conference room, a theater, a passenger compartment, a supermarket, or a sales room. In the interior space, there is a path along which the cart can travel, for example, an aisle in the supermarket or in a bus. Other areas of the interior space may be inaccessible for the cart or only accessible with difficulty, for example, areas in which shelves or rows of seats are arranged. Typically, however, it is possible in interior spaces with a wide variety of spatial divisions to reach the vicinity of all areas to be disinfected via a path accessible to the cart.

The germicidal light source emits light that has an effect that kills germs or makes them harmless. Germs of all types, in particular bacteria, viruses, and fungi, can be affected by this effect. By irradiating with the germicidal light source, the desired disinfection can therefore ideally be achieved for any germ load. Preferably, a light source is used that is not dangerous for humans, i.e., in particular does not have a carcinogenic effect. However, this is not necessary if exposure of humans and, if applicable, other living creatures during use of the device can be avoided.

The drone is an unmanned aircraft that can move through the air in an environment of the cart in a targeted manner. It has an electric drive, typically with one or more rotors. Flight height, flight direction and flight speed can be controlled by a suitable control. To detect the spatial environment, the drone can have spatial detection sensors, in particular a LIDAR (light detection and ranging) system. The radius of action of the drone is limited basically by the electrical line with which the drone is connected to the cart. The length of this cable connection can be, for example, in the range from 1 m to 20 m, in particular in the range from 2 m to 5 m. Within its radius of action, the drone can move through the interior space in a targeted manner and, in doing so, subject surfaces, in particular located in the vicinity of the drone, to the light radiated by the germicidal light source fastened to the drone.

Aside from the wavelength, the disinfection effect depends primarily on the duration of exposure and intensity of the emitted light. Both factors can be influenced by the flight speed of the drone and by the distance maintained from the surfaces to be disinfected. In addition to the surface disinfection, a disinfection of the room air can be effected, including in connection with aerosols. This is particularly advantageous in connection with small aerosol droplets with a diameter of less than approximately 3 µm, because they cannot be removed from the room air with a HEPA filter.

An important characteristic of the invention is the combination of a cart and a drone, the electric drive and light source of which are supplied with energy via a cable. This combination offers the advantage of being able to reach practically all relevant surfaces even in complexly designed, angled, or confined interior spaces, and without a complex and potentially fragile mechanical system for positioning the light source. Also, no special adaptation of the device is required for different interior spaces, so that the device can be used particularly flexibly. In this case, the energy for the drone is supplied by the cart, so that powerful light sources that are required for fast disinfection can be used without limiting the possible duration of use.

It is understood that the device is not limited to a single drone connected to the cart. In particular, two, three, four or more than four drones can be used, which are each connected to the cart with a cable and on each of which at least one germicidal light source is fastened. As a result, multiple surfaces and/or different areas of the same surface can be irradiated at the same time. When using multiple drones, they can also be connected to the cart with cables of different lengths. For example, a first drone can have a shorter cable, which limits its use to a correspondingly small circle around the cart, and a second drone can have a longer cable and can be used at a greater distance from the cart, for example, in an annular surrounding area that adjoins the outside of the circle covered by the first drone.

Additional germicidal light sources can be arranged on the cart and can irradiate surfaces in a closer environment of the cart independently of the drone. For example, in a lower area of the cart, one or more germicidal light sources can be arranged which are aimed at a floor. Alternatively or additionally, one or more germicidal light sources can be fastened to the cart and radiate their light in a lateral direction and/or upwards. As a result, a complete disinfection of the interior space can possibly take place even faster.

In one embodiment, the drone is a multicopter, i.e., it has multiple rotors responsible for lift and propulsion, in particular a quadrocopter with four rotors. Multicopters and especially quadrocopters have proven themselves due to their ability to hover in place and due to their good controllability. These properties can be particularly important for a cable-bound drone. They have a sufficient load-bearing capacity so that powerful and relatively large and/or heavy light sources can also be used, even in combination with relatively long cables.

In one embodiment, the interior space is an aircraft cabin and the path is an aisle of the aircraft cabin. All areas of the aircraft cabin are easily accessible from the aisle of the aircraft cabin. In particular, all seats can be reached from an aisle with few steps. At the same time, the spatial conditions in the aircraft cabin are confined and there are a plurality of differently oriented surfaces that are touched by the passengers. This includes the sitting surfaces as well as the backrests and armrests of the seats, but also the rear sides, facing the passengers, of the seats in front of them, seat belts and seat belt buckles, operating elements for air nozzles, folding tables, light switches, and a service personnel call button. In this configuration, use of the device according to the invention is particularly advantageous. In particular, the drone can be connected to the cart with a relatively short cable and still disinfect all relevant aircraft cabin areas. Since rows of seats are typically arranged on both sides of an aisle of an aircraft cabin, an embodiment with two drones is particularly practical. In particular a cabin trolley can be used as the cart, in particular a cabin trolley with aviation approval (for example, ATLAS or SAE-AS 8056) in half-size or full-size format. In this case, the cart or, respectively, the entire device can be transported on board the aircraft. The disinfection of the aircraft cabin can then begin after the aircraft has landed, in particular as soon as the passengers have left the aircraft cabin.

In one embodiment, the light source is a UVC light source. The germicidal effect of UVC light has been proven in practice. Light sources in the far UVC range, i.e., with a maximum intensity in the wavelength range from approximately 240 nm to approximately 100 nm, in particular in the range from approximately 230 nm to approximately 200 nm, for example at approximately 222 nm, can be used particularly advantageously. UVC light in this wavelength range offers a high disinfection effect and is at the same time harmless for larger living creatures, because it does not penetrate so deeply into the skin that the tissue is damaged. In particular, a carcinogenic effect can be largely avoided.

In one embodiment, the UVC light source is an excimer lamp, in particular a krypton chloride excimer lamp. Such lamps have a wavelength of approximately 222 nm. They are offered, for example, by the company Far UV Technologies, Inc. from Kansas City. In principle, any other UVC light sources can also be used, for example, mercury vapor lamps or UVC LEDs. Excimer lamps are characterized by a high light intensity and advantageous radiation properties.

In one embodiment, the germicidal light source has two tubular illuminants arranged parallel next to each other. This arrangement is particularly favorable for fastening to a drone and has good radiation characteristics for disinfecting surfaces.

In one embodiment, two rotors of the drone are arranged on each of two opposing ends of the arrangement of the illuminants. The drone is accordingly in particular a quadrocopter. The rectangle formed by the four rotors can be formed by the illuminant arrangement and/or be filled by it completely or partially. In this way, the illuminants can contribute to the structural stability of the drone, which enables a particularly light design.

In one embodiment, the plane defined by the two parallel, tubular illuminants is arranged tilted at an angle in the range of 5° to 45° relative to a rotor plane. The rotors of the drone each rotate in a plane which is horizontal (at least when hovering). The arrangement of the illuminant arrangement tilted relative to this horizontal can create in particular a main irradiation direction that faces downward and slightly forward, which is particularly favorable for an effective surface disinfection, for example, of sitting surfaces, which are oriented basically horizontally.

In one embodiment, at least one row of seats is arranged in the interior space and the drone has an electronic control, which is configured to control the drone automatically such that the drone moves at a specified speed along the at least one row of seats. In addition, the control can be configured to maintain a specified distance from the sitting surfaces and/or from the backrests of the seats of the row of seats during the movement along the at least one row of seats. For this purpose, the drone can have the previously mentioned spatial detection sensors and/or a camera system that detects the arrangement of the seats, and/or a distance measuring apparatus. By controlling the drone in a targeted manner along the row of seats, it is ensured that all seats of the row of seats are irradiated with a dose of the germicidal light that is sufficient for the sought-after disinfection. The row of seats can be arranged, for example, in a movie theater or theater or in an aircraft cabin.

In one embodiment, the cart has an extendable mast, which in a retracted position is accommodated inside the cart and in an extended position projects upwards out of the cart, wherein the electrical line is connected to the mast. For error-free flight of the drone, it can be advantageous if the electrical line is fastened at a relatively high height on the cart, for example, at a height in the range from 1.20 m to 1.80 m and thus, for example, above the headrests of typical seats. The mast serves this purpose, wherein the electrical line is preferably attached to an upper end of the mast and/or is guided out of the mast. A particular advantage of the extendable mast is that the mast is accommodated completely inside the cart in its retracted position, so that the cart can be stowed in a space-saving manner. This is important, for example, when the device should be brought on board an aircraft.

In one embodiment, the device has an electric travel drive and an electronic control for the cart, wherein the control is configured to control the cart autonomously along the path. In principle, the device can also be used with a cart without its own travel drive, for example, if the cart is pushed or pulled along the path by an operating person. With its own travel drive for the cart, no operating person is required for this. This applies in particular in connection with the autonomous control for the cart. Unlike in the case of, for example, a remote control for the cart, which is also possible, no operating person is then required to control the cart. In order for the cart to find its way autonomously, it can be equipped with a suitable navigation system, in particular with a camera system. The camera system can detect an environment of the cart and recognize the path by itself. Alternatively or additionally, the path to be traveled can have a marking, for example, optically detectable path marks, induction cables, or magnetic markings. When traveling along the path, the autonomous control of the cart can in particular select the travel speed or suitable intermediate stops such that sufficient time for the disinfection of the adjacent areas, for example, the mentioned rows of seats, is available to the drone.

In one embodiment, the device comprises a power cable, which is connected to the cart and has a plug for connecting to a supply network, in particular an on-board electrical system of an aircraft. In principle, the cart can be supplied in any way with electrical energy for the drone and possibly for additional elements such as its own electric travel drive, a control, etc. For example, a correspondingly dimensioned, rechargeable battery can be integrated into the cart. For many purposes, in particular for use on board an aircraft, however, a battery supply is problematic, because larger batteries cannot be readily taken on board an aircraft. Additional difficulties can result with regard to charging such a battery. The supply of the cart from a supply network via a power cable is a practicable alternative, with which in particular powerful germicidal light sources and electric drives for the drone can also be simply and reliably supplied. After stowing the power cable, there are also no particular risks with regard to transport on board an aircraft. The supply network can be an in-house supply network available in the interior space in question (for example, 230 V alternating current) or another suitable supply network. The on-board electrical system of the aircraft can have, for example, a nominal voltage of 28 V DC or a nominal voltage of 115 V AC or 230 V AC at a frequency of 400 Hz or 800 Hz.

In one embodiment, the device has a coiling device for the power cable, which is designed to pull the cart along a specified travel path by coiling the power cable. In certain use cases, the cart can be moved forward particularly easily as a result. This applies, for example, in an aircraft cabin having what is known as an in-cabin belt system.

In one embodiment, the device has a coiling device for the power cable, wherein the electronic control for the cart is designed to coil and uncoil the power cable automatically during travel depending on a travel speed and/or travel direction. Through such an automatic coiling and uncoiling of the power cable, the cart can possibly even travel complicated and/or longer paths without the power cable presenting particular difficulties. For example, the cart can travel along a center aisle through an aircraft cabin, beginning, for example, all the way at the front by the cockpit where the plug is connected to the on-board electrical system of the aircraft. In this case, the power cable is uncoiled on the path to the rear end of the aircraft cabin and coiled on the return path. This can be done fully automatically so that the cabin personnel simply have to put the device into operation in the vicinity of the cockpit and stow it there again after disinfection is completed.

In one embodiment, the device has a travel drive module on which the cart can be arranged and fastened, wherein the travel drive module has in particular a crawler track. In this special combination, the cart has rollers or wheels on one side, on which it can be pushed or pulled by hand, for example, in order to stow the cart on board an aircraft cabin, for example, in a holder for a cabin trolley. In addition, there is a travel drive module, on which the cart can be driven along the path. The travel drive module can have, for example, multiple, partially steered wheels for this purpose. In particular, this can be a crawler track, which enables particularly error-free operation at the relatively low travel speeds, since it can overcome, for example, smaller obstacles particularly easily.

In one embodiment, the travel drive module has a ramp on which the cart can be driven by hand onto the travel drive module. For this purpose, the travel drive module can be placed on the floor. Then, the cart is driven by hand over the ramp onto the travel drive module and fixed in the desired position on the travel drive module. For further travel, the wheels of the cart are then no longer required.

In one embodiment, the cart has an accommodation compartment for the drone and/or an accommodation compartment for the travel drive module, so that the drone including the electrical supply line or, respectively, the travel drive module can be stowed inside the cart. This is particularly important, for example, on board an aircraft in order to be able to stow the cart in a standardized holder. At the same time, the drone and/or the travel drive module are optimally protected during transport of the device.

### BRIEF DESCRIPTION OF THE SEVERAL VIEWS OF THE DRAWINGS

The invention is explained in greater detail below with reference to exemplary embodiments shown in figures. In the drawings:

FIG. 1: shows a device for disinfecting an interior space, the elements of which are stowed inside its cart,

FIGS. 2 to 7: show the device from FIG. 1 in different states of assembly,

FIG. 8: shows the device from FIG. 1 during disinfection of rows of seats of an aircraft cabin,

FIG. 9: shows a drone of the device from FIG. 1 in a view from above,

FIG. 10: shows the drone from FIG. 9 in a view from the side,

FIG. 11: shows the drone from FIG. 9 in a perspective representation,

FIG. 12: shows another device for disinfecting an interior space during disinfection of rows of seats of an aircraft cabin.

### DETAILED DESCRIPTION OF THE INVENTION

All figures are schematic and refer by way of example to an application of the device in an aircraft cabin.

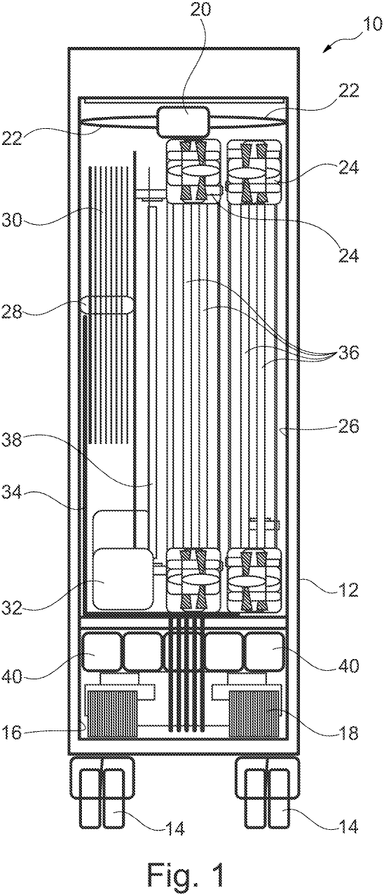

FIG. 1 shows a device 10 for disinfecting an aircraft cabin, which as a cart has an aviation-approved cabin trolley 12 with a cuboid, upright body and four wheels 14 arranged on its underside. Such cabin trolleys 12 are often used to supply passengers with snacks and drinks and are pushed or pulled by hand through an aisle of the aircraft cabin for this purpose. In the state of the device 10 shown in FIG. 1, all elements required for disinfection are located within the body so that the entire device 10 can be stored like a usual cabin trolley 12 in a stowage space provided for this purpose on board the aircraft.

An accommodation compartment 16 in which a travel drive module 18 is stored is located in a lower area of the body of the cabin trolley 12. For this purpose, the accommodation compartment 16 is adapted such that the travel drive module 18 is held securely therein, for example, by means of an exact fit and/or a special fastening apparatus for the travel drive module 18.

An upwardly extendable mast 20, only an indication of which can be seen, from which two electrical lines 22 (only partially visible in FIG. 1) extend, is located in an upper area of the body of the cabin trolley 12. These two electrical lines 22 each lead to a drone 24. The two drones 24 are stored in an additional accommodation compartment 26 in the body of the cabin trolley 12. The additional accommodation compartment 26 is also adapted such that the drones 24 are held securely therein, for example, by means of an exact fit and/or a special fastening apparatus for the two drones 24.

A coiling device 28, on which the power cable 30 is coiled, is located laterally of the additional accommodation compartment 26. The device 10 can be connected to an on-board electrical system of an aircraft via the power cable 30, which has a plug on its free end (not shown).

Inside the cabin trolley 12, the device 10 also has two transformers 32, through which the individual elements of the device 10 are supplied with electrical energy from the on-board electrical system of the aircraft. In this regard, additional electrical lines 34 are indicated in FIG. 1, which lead from the coiling device 28 for the power cable 30 to the transformers 32 or, respectively, from there to the travel drive module 18.

As FIG. 1 also shows, the device 10 has a number of germicidal light sources: Each of the two drones 24 has two germicidal light sources 36. Another germicidal light source 38 is assigned to the extendable mast 20. Five additional germicidal light sources 40 are assigned to the travel drive module 18. All germicidal light sources 36, 38, 40 are securely stowed inside the cabin trolley 12. In the device 10 shown by way of example, each of the germicidal light sources 36, 38, 40 has a krypton chloride excimer lamp that emits UVC light with a wavelength of approximately 222 nm.

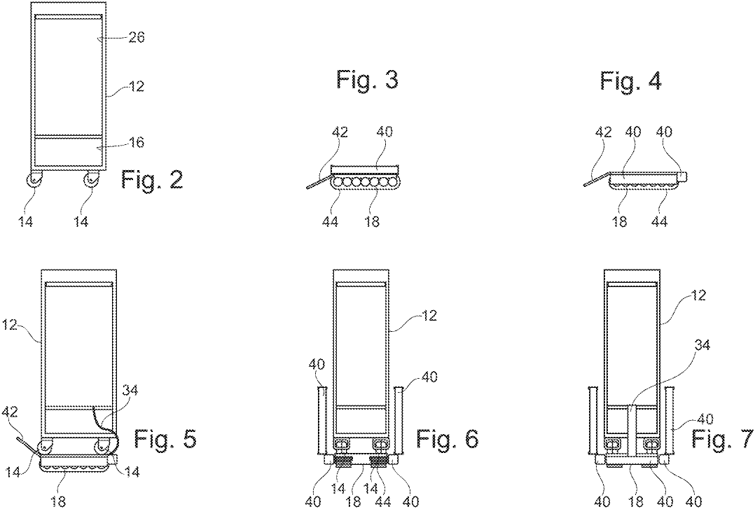

Additional details of the device 10, in particular how it can be transferred from the state shown in FIG. 1, in which all elements are stowed within the cabin trolley 12, to an operating state, are explained using FIGS. 2 to 7. FIG. 2 shows the cabin trolley 12 in a view from the side, wherein the elements arranged inside the cart are not shown. However, the two accommodation compartments 16 and 26 are clearly visible.

FIG. 3 shows the travel drive module 18, which has been removed from the accommodation compartment 16 together with the five additional germicidal light sources 40 and placed on the floor. A ramp 42 of the travel drive module 18, which has already been folded down, is clearly visible. The travel drive module 18 has a crawler track with two crawlers 44 arranged laterally next to each other.

FIG. 4 shows that in the next assembly step, three of the five additional germicidal light sources 40 have been arranged in their respectively provided positions on the travel drive module 18. This is done by a suitable folding apparatus or, for example, by simply plugging the additional germicidal light sources 40 into the holders provided for this on the travel drive module 18. As shown in FIG. 4, another germicidal light source 40 is then located at a low height above the floor at a front end of the travel drive module 18, so that, during travel, in particular the floor located below and in front of the travel drive module 18 is irradiated. Two of the additional germicidal light sources 40 have been arranged laterally on the travel drive module 18, also at a low height above the floor. They irradiate in particular floor areas laterally of the path along which the device 10 travels.

After the travel drive module 18 has been brought into the position shown in FIG. 4, the cabin trolley 12 can travel easily onto the travel drive module 18 via the ramp 42. Then the arrangement shown in FIG. 5 results, in which the cabin trolley 12 is located with its wheels 14 on the travel drive module 18. In this position, the cabin trolley 12 is fixed on the travel drive module 18. As indicated in FIG. 5, this can be done partially by folding up the ramp 42. In the simplest case, the wheels 14 of the cabin trolley 12 can be placed into precisely worked indentations on the upper side of the travel drive module 18. Alternatively or additionally, fixing the cabin trolley 12 to the travel drive module 18 with, for example, belts or clamping levers is possible.

FIG. 5 also shows that the travel drive module 18 is connected to the cabin trolley 12 or, respectively, the transformers 32 arranged therein via one of the additional lines 34 and thus can be supplied with electrical energy via the power cable 30.

In FIG. 6, the arrangement from FIG. 5 is shown in a view from behind, wherein, in addition to the three additional germicidal light sources 40 already described, two additional germicidal light sources 40 have been fastened in an upright arrangement laterally of a lower portion of the cabin trolley 12. In this position, they irradiate in particular the lateral surfaces, facing the aisle, of the adjacent seats when the device 10 travels along an aisle of an aircraft cabin.

FIG. 7 shows the arrangement from FIG. 6 in a view from the front, in which the view is aimed at the front side of the travel drive module 18 with the additional germicidal light source 40 fastened transversely there. The additional electrical line 34 that connects the travel drive module 18 to the cabin trolley 12 can also be seen.

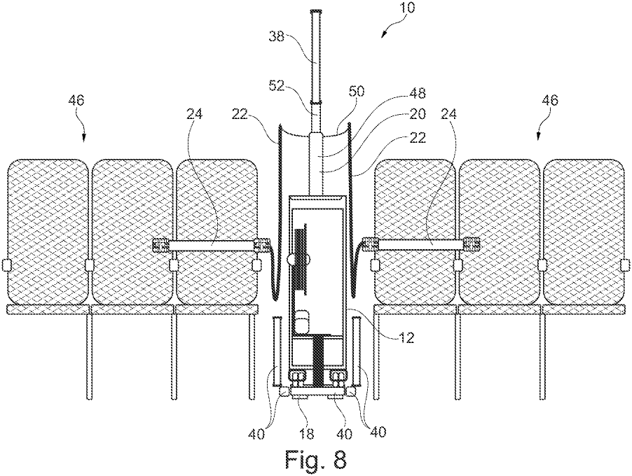

In FIG. 8, the device 10 can finally be seen in use, during the disinfection of two rows of seats 46 arranged on both sides of an aisle in an aircraft cabin. The lower part of the device 10 is located in the state explained in FIG. 7, with the cabin trolley 12 on the travel drive module 18 and the described arrangement of the additional germicidal light sources 40. In contrast to FIG. 7, the two drones 24 are now hovering on both sides of the cabin trolley 12. Moreover, the extendable mast 20 has been extended so that it projects upwards out of the cabin trolley 12.

It can be seen in FIG. 8 that the mast 20 is a telescopic mast, wherein the two electrical lines 22 are guided out of the mast 20 at the upper end of an outer telescopic portion 48. Starting from small, approximately horizontally arranged spacers 50 that protrude laterally out of the telescopic mast portion 48, the electrical lines 22 hang down freely and each lead to one of the two drones 24, forming a loop.

An inner telescopic mast portion 52 of the extendable mast 20 projects upwards out of the outer telescopic mast portion 48 and supports on its free end the additional germicidal light source 38, which faces vertically upwards. In this position, the additional germicidal light source 38 serves in particular to disinfect luggage compartments not shown in FIG. 8, which are located in the upper area of the aircraft cabin on both sides of the aisle.

With the aid of a control (not shown), the two drones 24 can each be moved in a targeted manner at a specified distance along the rows of seats 46 so that they irradiate each of the sitting surfaces and each of the backrests of the seats of the rows of seats 46 one after another until the desired disinfection effect has been achieved. For this purpose, the control executes the flight movement with a suitable, specifiable flight speed and a suitable, specifiable distance from the surfaces to be irradiated. It can be seen in FIG. 8 that the dimensions of the drones 24, in particular the length of the germicidal light sources 36 used, are adapted to the width of the individual seats: The illuminants have a length that is the same size or somewhat larger than a width of the seats. As a result, during a linear flight of the drone 24, one sitting surface and one backrest can be disinfected over their entire width.

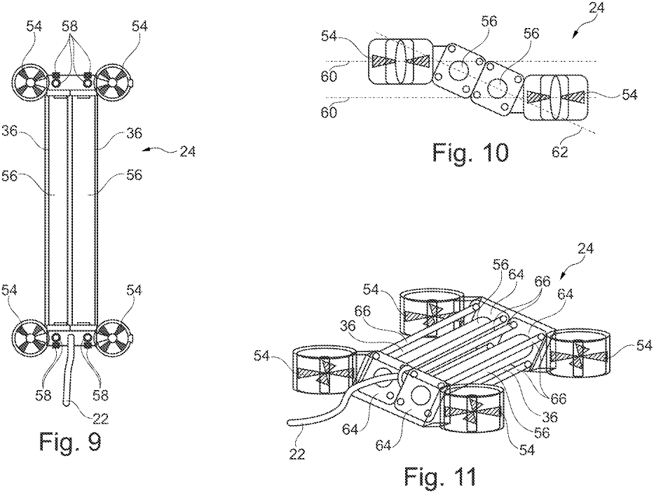

Additional details of the drones 24 can be seen more easily in FIGS. 9 to 11. In FIG. 9, a view from above, it can be seen that the drones 24 are quadrocopters with four rotors 54. The two germicidal light sources 36 have tubular illuminants 56 arranged parallel next to each other. Two of the rotors 54 of the drone 24 are arranged on each of the two opposing ends of this arrangement of the illuminants 56. Multiple sensors 58, with which an environment of the drone 24 can be detected during flight for the purpose of controlling the drone 24, can also be seen. The electrical line 22 is fastened to an end of the drone 24 between two rotors 54. It supplies both electric drives of the rotors 54 and the germicidal light sources 36 with electrical energy.

FIG. 10 shows the drone 24 from FIG. 9 in a view from the side. It can be seen that the rotors 54 each have a rotor plane 60 that is oriented approximately horizontally when hovering. The two tubular illuminants 56 that are arranged parallel define another plane 62, which is arranged tilted at an angle of approximately 15° relative to the rotor planes 60. The two rotor planes 60 are correspondingly offset in the vertical direction.

FIG. 11 shows a perspective view that once again illustrates the explained design of the drones 24. It can be seen that the germicidal light sources 36 each have one of the illuminants 56 along their longitudinal axis and a support structure around it, which in the example shown has two rectangular end plates 64 and four rods 66 connecting them to each other. This support structure protects the illuminant 56 from damage. At the same time, the germicidal light sources 36 form an essential element of a support structure of the drone 24.

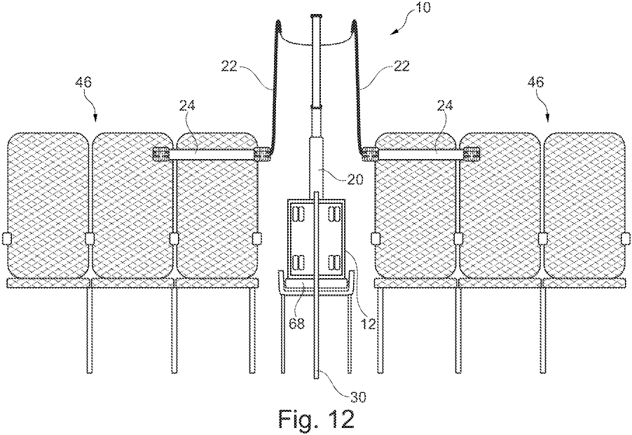

FIG. 12 shows another device 10 for disinfecting an aircraft cabin that also has a cabin trolley 12 as a cart and two drones 24. However, no travel drive module 18 creates the movement of the cabin trolley 12 along the aisle as in the previously described exemplary embodiment, but rather the cabin trolley 12 is arranged lying on a rear side on a transport belt 68 running along the aisle. The transport belt 68 is part of what is known as an in-cabin belt system, with which luggage can be transported along the aisle. In connection with the device according to the invention, the coiling device 28 and the power cable 30 of the cabin trolley 12 that is coiled with it is used to pull the device 10 along the transport belt 68. The connection of the drones 24 via electrical lines 22 takes place, as in the previous exemplary embodiment, via an extendable mast 20, which, however, is arranged on the cabin trolley 12 so that it projects from the cabin trolley 12 from a side located upwards in the lying position.

1. A device (10) for disinfecting an interior space comprising
a cart, which is configured to travel along a path through the interior space, and
a germicidal light source (36), characterized by
a drone (24), which has at least one electric drive for generating lift, wherein
the germicidal light source (36) is fastened to the drone (24) and
the drone (24) is connected to the cart via an electrical line (22) for supplying energy to the germicidal light source (36) and the electric drive.

2. The device (10) according to claim 1, characterized in that the interior space is an aircraft cabin and the path is an aisle of the aircraft cabin.

3. The device (10) according to claim 1, characterized in that the germicidal light source (36) is a UVC light source, in particular with a maximum intensity in the wavelength range from 240 nm to 100 nm.

4. The device (10) according to claim 1, characterized in that the UVC light source is an excimer lamp, in particular a krypton chloride excimer lamp.

5. The device (10) according to claim 1, characterized in that the germicidal light source (36) has two tubular illuminants (56) arranged parallel next to each other.

6. The device (10) according to claim 5, characterized in that two rotors (54) of the drone (24) are arranged on each of two opposing ends of the arrangement of the illuminants (56).

7. The device (10) according to claim 5, characterized in that a plane (62) defined by the two parallel, tubular illuminants (56) is arranged tilted at an angle in the range of 5° to 45° relative to a rotor plane (60).

8. The device (10) according to claim 1, characterized in that at least one row of seats (46) is arranged in the interior space and the drone (24) has an electronic control, which is configured to control the drone (24) automatically such that the drone (24) moves at a specified flight speed along the at least one row of seats (46).

9. The device (10) according to claim 1, characterized in that the cart has an extendable mast (20), which in a retracted position is accommodated inside the cart and in an extended position projects upwards out of the cart, wherein the electrical line (22) is connected to the mast (20).

10. The device (10) according to claim 1, characterized in that the device (10) has an electric travel drive and an electronic control for the cart, wherein the control is configured to control the cart autonomously along the path.

11. The device (10) according to claim 1, characterized in that the device (10) comprises a power cable (30) that is connected to the cart and has a plug for connecting to a supply network, in particular an on-board electrical system of an aircraft.

12. The device (10) according to claim 11, characterized in that the device (10) has a coiling device (28) for the power cable (30), which is designed to pull the cart along a specified travel path by coiling the power cable (30).

13. The device (10) according to claim 11, characterized in that the device (10) has a coiling device (10) for the power cable (30), wherein the electronic control for the cart is designed to coil and uncoil the power cable (30) automatically during travel depending on a travel speed and/or travel direction.

14. The device (10) according to claim 1, characterized in that the device (10) has a travel drive module (18), on which the cart can be arranged and fastened, wherein the travel drive module (18) has in particular a crawler track.

15. The device (10) according to claim 1, characterized in that the cart has an accommodation compartment (26) for the drone (24) and/or an accommodation compartment (16) for the travel drive module (18), so that the drone (24) including the electrical supply line (22) or, respectively, the travel drive module (18) can be stowed inside the cart.
